(19) United States
(12) Patent Application Publication
(10) Pub. No.: US 2019/0365275 A1
(43) Pub. Date: Dec. 5, 2019

(54) SEMICONDUCTOR DEVICE, MEASUREMENT SYSTEM AND MEASUREMENT METHOD

(71) Applicant: LAPIS SEMICONDUCTOR CO., LTD., Kanagawa (JP)

(72) Inventor: Teruaki UEHARA, Kanagawa (JP)

(21) Appl. No.: 16/423,237

(22) Filed: May 28, 2019

(30) Foreign Application Priority Data
Jun. 4, 2018 (JP) 2018-107204

(51) Int. Cl. A61B 5/053; A61B 5/00

(52) U.S. Cl. CPC A61B 5/0537; A61B 2560/0223; A61B 5/443

(57) ABSTRACT

A semiconductor device including: a signal source that generates a sine wave signal; an output section that outputs a measurement signal corresponding to the sine wave signal to a test subject via a first electrode; an input section that receives, as an input signal, the measurement signal that has passed through the test subject and been input via a second electrode; a first calculation device that calculates correlation values between the sine wave signal and the input signal; and a second calculation section that, based on the correlation values, calculates a bioelectrical impedance of the test subject.

### CROSS-REFERENCE TO RELATED APPLICATION

This application is based on and claims priority under 35 USC 119 from Japanese Patent Application No. 2018-107204, filed on Jun. 4, 2018, the disclosure of which is incorporated by reference herein.

### BACKGROUND

### Technical Field

The present disclosure relates to a semiconductor device, a measurement system, and a measurement method, that are used to measure bioelectrical impedance.

### Related Art

Conventionally, Japanese Patent Application Laid-Open (JP-A) No. 2001-212101 is known as a disclosure relating to bioelectrical impedance. An electrical characteristics measurement device disclosed in JP-A No. 2001-212101 is provided with a measurement signal generating section that generates a digital waveform, a measurement signal supply section that has a conversion section that converts this digital waveform into an analog waveform, and that supplies the analog waveform signal to a test subject via a first and a second electrode that are attached, in such a way that they can be supplied with power, at two predetermined, mutually separated locations on a surface portion of the test subject, a current measurement section that measures a current value of the analog waveform signal supplied to the test subject, a voltage measurement section that measures voltage values generated between the two predetermined, mutually separated locations on the surface portion of the test subject, and a calculation section that calculates a bioelectrical impedance between the surface portions of the test subject using the current values and voltage values measured respectively by the current measurement section and the voltage measurement section, and calculates a bioelectrical impedance that is to be determined or physical quantities based on a bioelectrical impedance.

An object of the disclosure of JP-A No. 2001-212101 is to provide an electrical characteristics measurement device that is suitable for shortening the measurement time while avoiding the effects of external noise on the low frequency side, and for measuring bioelectrical impedance, and for measuring the states of body fat and the distribution of body water. In measurements of biomedical impedance of this type, generally, a measurement method known as a BI (Bioelectrical Impedance) method is used, and the measured body water distribution is used, for example, in a skin sensor, while the measured body fat is used, for example in a body fat meter and the like.

In the case of a skin sensor, the impedance of a test subject is measured by placing a pair of electrodes spaced approximately 1 cm apart in contact with the test subject's skin, and supplying a weak current to the electrodes. The moisture content of the skin is then determined by comparing this impedance to an impedance/moisture content (%) table (i.e., a conversion table). In this case, the impedance/moisture content (%) table is prepared in advance prior to the measurement being taken. In contrast, in the case of a body fat meter, body fat percentage is obtained by supplying a weak current through the trunk of a test subject, either from one leg to the other leg, or from one hand to the other hand thereof, and measuring the resulting impedance. This measured impedance is then compared to plural body fat (%) percentage/impedance tables (i.e., conversion tables) that have been prepared in advance for a variety of heights and body weights.

As described above, in conventional skin sensors, information relating to the skin is obtained from the impedance between electrodes spaced approximately 1 cm apart. Due to the above, cases described below may occur.

(1) Since these sensors can only be used by placing electrodes in contact with skin, only information about the surface of the skin can be obtained. In other words, information about, for example, a collagen layer which is underneath the dermis and is crucial to healthy skin cannot be obtained.
(2) If the contact surface area between the skin and the electrodes changes, then the measured impedance also changes. This is due to that the impedance is inversely proportional to the size of the surface area between the skin and the electrodes. In this case, it is not possible to use previously prepared impedance/moisture content (%) tables. In other words, the state of the moisture content is unclear.

Furthermore, as described above, conventional body fat meters obtain a body fat percentage by supplying a weak current through the trunk of a test subject and measuring the resulting impedance. This measured impedance is then compared to plural body fat (%) percentage/impedance tables that have been prepared in advance for a variety of heights and body weights. In a conventional measurement method, acquired impedance values are used without any modification. Due to this, contact resistance between the electrodes and the body has a considerable effect on the measurement results, and thus, obtaining accurate measurement values may not be possible. Conventionally, in order to solve the above, two electrodes have been used at both the entry and exit points of the weak current, and the impedance has been measured after contact resistance has been cancelled out by means of calculation.

In addition, because measured impedance values are used directly, the length of the path of the current also has an effect on the measurement results. In order to solve this, conventionally, information such as the height, body weight, gender, age, and the like of a test subject is input into the measurement device prior to measurement, and measurement results containing fewer errors are obtained by selecting the aforementioned conversion tables. In other words, in order to obtain a more accurate body fat percentage, it has been necessary to input personal information into the measurement device, and because of recent trends regarding personal information, this has often been a problem, for example, for people who are sensitive about their personal information.

In consideration of the above, a measurement device and a measurement method that may enable body composition data, such as skin moisture content and the like, to be correctly measured, even when there is a change in a form of contact between skin and contacting terminals, are required. In other words, a measurement device and a measurement method that may measure underneath the dermis, even when there is a change in a form of contact between skin and contacting terminals, are desired in a skin sensor. Moreover, a measurement device and a measurement method that may measure a body fat percentage without having to use any reference data (e.g., height, body weight, or the like) are desired in a body fat meter.

### SUMMARY

The present disclosure provides a semiconductor device, a measurement system, and a measurement method that may accurately measure biometric information, without having to refer to any physical data, even when there is a change in a form of contact between a surface portion and contact terminals.

A first aspect of the present disclosure is a semiconductor device including: a signal source that generates a sine wave signal; an output section that outputs a measurement signal corresponding to the sine wave signal to a test subject via a first electrode; an input section that receives, as an input signal, the measurement signal that has passed through the test subject and been input via a second electrode; a first calculation device that calculates correlation values between the sine wave signal and the input signal; and a second calculation section that, based on the correlation values, calculates a bioelectrical impedance of the test subject.

A second aspect of the present disclosure is a measurement system including: the semiconductor device according to the first aspect, a first electrode that, outputs to a test subject, a measurement signal corresponding to the sine wave signal from the output section; and a second electrode that receives, as an input signal, the measurement signal that has passed through the test subject.

A third aspect of the present disclosure is a measurement method that uses a measurement system including plural signal sources that respectively generate sine wave signals having mutually different frequencies, a first electrode that outputs, to a test subject, a measurement signal corresponding to a signal obtained by adding plural the sine wave signals, and a second electrode that receives, as an input signal, the measurement signal that has passed through the test subject, the measurement method including: calculating correlation values between each one of the sine wave signals and the input signal; calculating a bioelectrical impedance of the test subject using the plural correlation values; separating the bioelectrical impedance into a resistance component and a capacity component; and measuring at least one of a state of the body composition of the test subject, by using a ratio of the resistance components, or a ratio of the capacity components measured by using each one of two frequencies, or a skin moisture content of the test subject, by using the ratio of the capacity components measured by using each one of the two frequencies.

According to the above aspects, a semiconductor device, a measurement system, and a measurement method of the present disclosure may accurately measure biometric information, and without having to refer to any physical data, even when a configuration of a surface portion that is in contact with contact terminals changes.

### BRIEF DESCRIPTION OF THE DRAWINGS

Exemplary embodiments of the present disclosure will be described in detail based on the following figures, wherein:

FIG. 1 is a block diagram illustrating a structure of a measurement system according to an exemplary embodiment;

FIG. 2 is a block diagram illustrating a structure of a semiconductor device according to an exemplary embodiment;

FIG. 3 is a portion of a flow chart illustrating a flow of processing of a measurement program according to an exemplary embodiment;

FIG. 4 is a portion of a flow chart illustrating a flow of processing of a measurement program according to an exemplary embodiment;

FIG. 5 is a view illustrating a calculation model of a measurement system according to an exemplary embodiment; and

FIG. 6 is a view illustrating a skin moisture calculation performed using the measurement system according to an exemplary embodiment.

### DETAILED DESCRIPTION

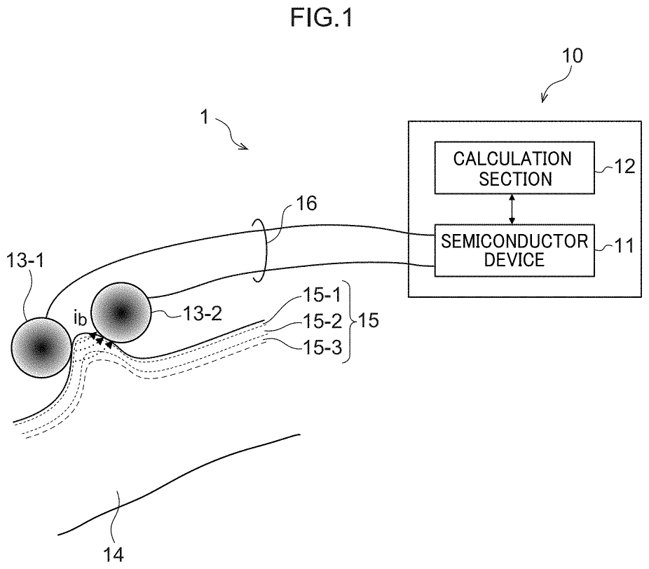

Hereinafter, an exemplary embodiment for implementing the present disclosure will be described in detail with reference to FIG. 1 through FIG. 6.

FIG. 1 illustrates an overall structure of a measurement system 1 according to the present exemplary embodiment in a state of adhesion to an arm 14. As illustrated in FIG. 1, the measurement system 1 is provided with a device main body 10, electrodes (i.e., skin contact terminals) 13-1 and 13-1 (hereinafter, described as ‘electrodes 13’ when referred to collectively), and wiring 16. The device main body 10 includes a semiconductor device 11 and a calculation section 12 (both are described below). The device main body 10 a principal portion for measuring bioelectrical impedance.

The electrodes 13-1 and 13-1 are a pair of electrodes that are each formed in a substantially spherical shape. Because of their spherical shape, as illustrated in FIG. 1, the electrodes 13-1 and 13-2 are able to be positioned on either side of (i.e., pinching) a fold of skin. By positioning the electrodes such that they are pinching a fold of skin, using the measurement system 1 according to the present exemplary embodiment, it is possible to measure a deep portion of skin tissue.

As illustrated in FIG. 1, skin 15 is formed by an epidermis 15-1, a dermis 15-2, and subcutaneous tissue 15-3. In a conventional measurement device that uses contact-type electrodes, the measurement current only reaches the depth of the epidermis 15-1 or the dermis 15-2. Because of this, it is difficult to measure the bioelectrical impedance of deep portions (i.e., the subcutaneous tissue 15-3) of the skin 15. In contrast, since the electrodes 13 according to the present exemplary embodiment are placed on either side of a fold of the skin 15, as illustrated in FIG. 1, a measurement current ib can be supplied easily as far as the subcutaneous tissue 15-3. Because of this, in the measurement system 1, in spite of a simple structure being used, measurement of the impedance of deep portions (i.e., the subcutaneous tissue 15-3) of the skin 15, which has a close connection to moisture and fat levels, may be performed easily.

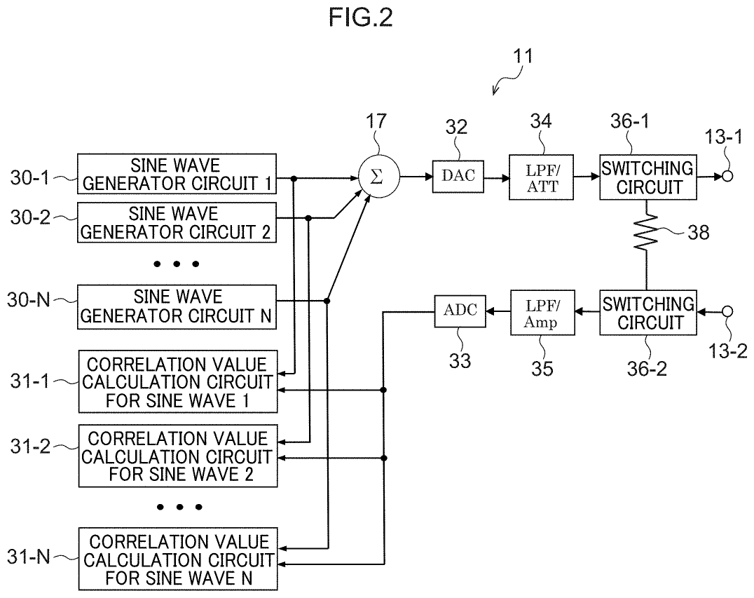

Next, the semiconductor device 11 according to the present exemplary embodiment will be described with reference to FIG. 2. As illustrated in FIG. 2, the semiconductor device 11 is formed so as to include plural (in FIG. 2, N number are illustrated) sine wave generator circuits 30-1, 30-2, . . . , 30-N (hereinafter, described as ‘sine wave generator circuits 30’ when referred to collectively), plural (in FIG. 2, N number are illustrated) correlation value calculation circuits 31-1, 31-2, . . . , 31-N (hereinafter, described as ‘correlation value calculation circuits 31’ when referred to collectively), a DAC (Digital Analog Converter circuit) 32, an adder circuit 17, an ADC (Analog Digital Converter circuit) 33, an LPF (Low Pass Filter)/ATT (Attenuator) 34, an LPF/Amp (Amplifier) 35, and switching circuits 36-1 and 36-2 (hereinafter, described as ‘switching circuits 36’ when referred to collectively).

The sine wave generator circuits 30 are circuits that generate sine wave signals in plural, mutually different frequencies. The respective frequencies of each sine wave generator circuit 30, as well as the total number thereof are not particularly limited.

The adder circuit 17 is a circuit that digitally adds together (i.e., superimposes) the sine wave signals generated by the respective sine wave generator circuits 30, and outputs the result as an added sine wave signal.

The DAC 32 converts the added sine wave signal, which is in a digital format, added by the adder circuit 17, into an analog signal.

The LPF/ATT 34 is formed by a circuit that functions as a low-pass filter and a circuit that functions as an attenuator. The LPF/ATT 34 blocks high-frequency components in added sine wave signals from the DAC 32, and performs level (i.e., amplitude) adjustment of signals (i.e. measurement signals) output from the electrode 13-1. Note that, in the present exemplary embodiment, the measurement signal is formed by a current signal. However, the present disclosure is not limited to this and the measurement signal may instead be formed by a voltage signal.

The switching circuit 36-1 and the switching circuit 36-2 operate in mutual cooperation, and switch a connection destination of the device main body 10 to either an electrode or to a reference resistor 38. The reference resistor 38 (described below) is a resistor whose resistance value is already known, and which is externally attached in the present exemplary embodiment. Naturally, the reference resistor 38 may be formed as part of the semiconductor device 11.

The LPF/Amp 35 is formed by a circuit that functions as a low-pass filter and a circuit that functions as an amplifier. The LPF/Amp 35 blocks high-frequency components in measurement signals transmitted through a test subject and input from the electrode 13-2, and amplifies the measurement signals to a predetermined level (i.e., amplitude).

The ADC 33 converts measurement signals, which are in an analog format, from the LPF/Amp 35, into digital signals.

The correlation value calculation circuits 31-1, 31-2, . . . , 31-N use the respective sine wave generator circuits 30-1, 30-2, . . . , 30-N as reference signals, and calculate correlation values between these reference signals and the measurement signals from the ADC 33. In other words, the calculation of the correlation values is performed for each individual frequency of the sine wave generator circuits 30. The N number of correlation values, calculated by the correlation value calculation circuits 31, are output to the calculation section 12, and are used in calculations performed by the calculation section 12. Here, the correlation values according to the present exemplary embodiment are autocorrelation coefficients, wherein autocorrelation is a scale that measures the degree to which one time-domain signal approximates another time-domain signal obtained by transforming the one time-domain signal. In the present exemplary embodiment, correlation values are used to detect measurement signals that have passed through a test subject and become buried in noise. Note that the calculation section 12 is an example of a ‘first calculation section’ and a ‘second calculation section’ according to the present disclosure.

The calculation section 12 is a calculation circuit that calculates impedance (i.e., bioelectrical impedance) values, biological resistance values, and capacity values using data from the correlation value calculation circuits 31. For example, a microcomputer is used for the calculation section 12, and the calculation section 12, in this case, is formed so as to include a CPU (Central Processing Section), ROM (Read Only Memory), and RAM (Random Access Memory), and the like (none of these are illustrated in the drawings). Note that, in the present exemplary embodiment, the semiconductor device 11 and the calculation section 12 are mutually independent of each other. However, the present disclosure is not limited thereto. It is also possible to form the calculation section 12 as an internal part of the semiconductor device 11 so that the semiconductor device 11 is formed as a single chip.

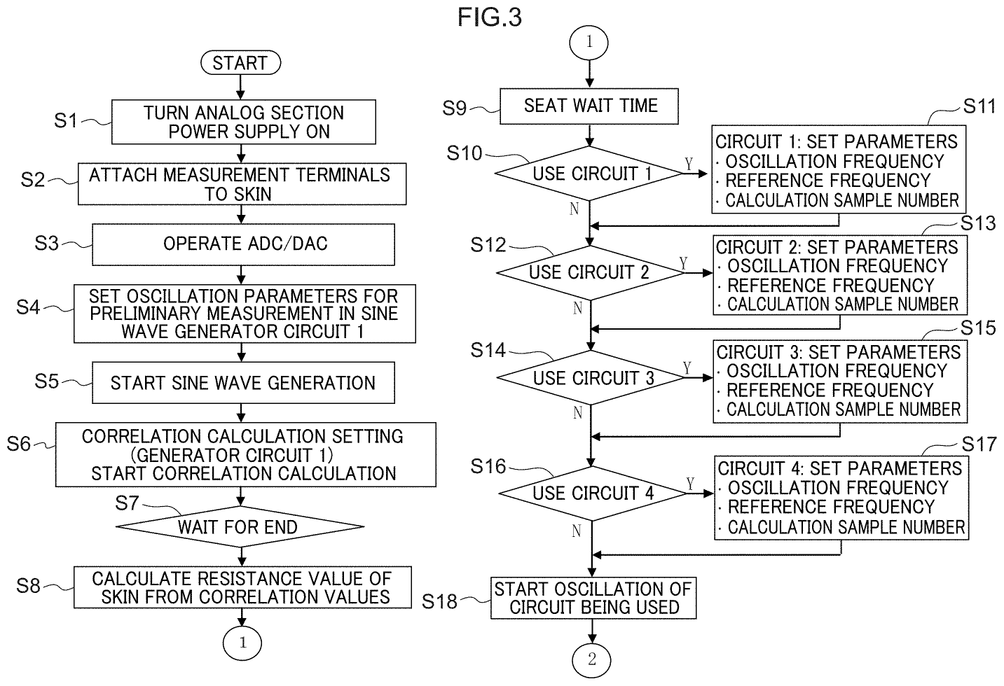

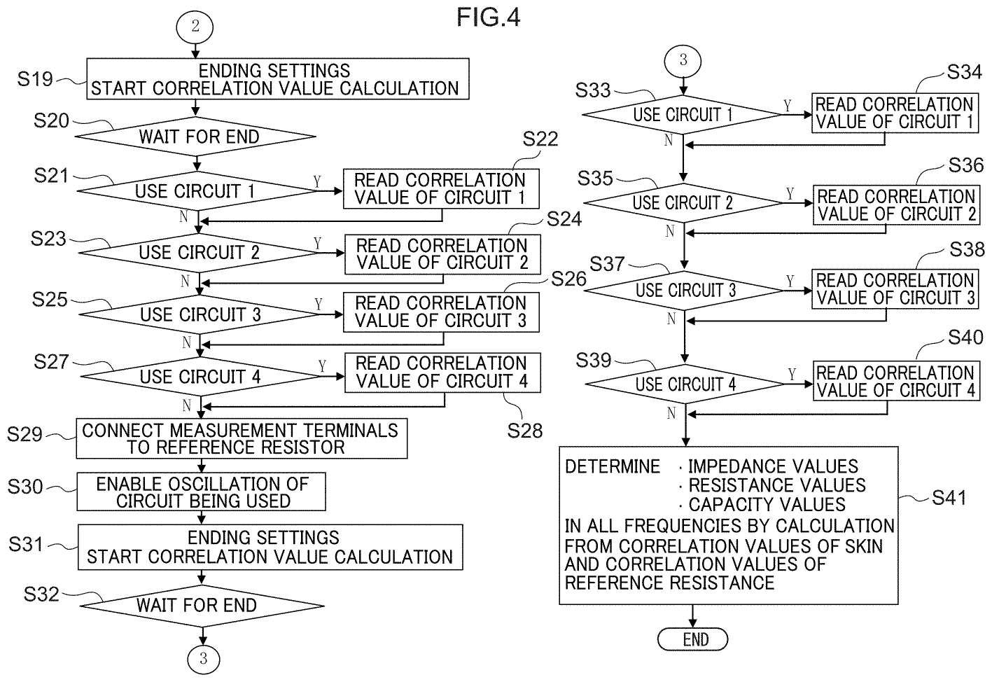

Next, measurement processing executed by the measurement system 1 according to the present exemplary embodiment will be described with reference to FIG. 3 and FIG. 4. FIG. 3 and FIG. 4 are flowcharts illustrating flows of processing performed by a measurement program according to the present exemplary embodiment. In the measurement system 1 according to the present exemplary embodiment, as a result of a measurement processing start command being given via a UI (User Interface) section or the like (not illustrated in the drawings), a CPU (not illustrated in the drawings) provided internally in the calculation section 12 executes the processing illustrated in FIG. 3 and FIG. 4 by reading this measurement program that is stored in a storage section such as ROM or the like, and expanding the program in RAM or the like. Note that, in the present exemplary embodiment, a structure in which four sine wave generator circuits 30 that each generate sine waves having mutually different frequencies are used, has been described as an example. In addition, in the present exemplary embodiment, a mode in which the measurement system 1 is made to function as a skin sensor has been described as an example.

As illustrated in FIG. 3, in step S1, a power supply of an analog section is tuned on. The analog section according to the present exemplary embodiment refers to a portion from an output of the DAC 32 in FIG. 2 to an input of the ADC 33.

In step S2, measurement terminals are attached to ‘skin’. In other words, the electrodes 13 are attached to the skin of a test subject, and the switching sections 36 are switched to the electrode 13 side. At this time, the pair of electrodes 13-1 and 13-2 are attached to pinch the skin from two sides.

In step S3, the ADC 33 and the DAC 32 are operated.

In step S4, oscillation parameters for preliminary measurement are set in the sine wave oscillator circuit 30-1. The preliminary measurement according to the present exemplary embodiment refers to a measurement in which an approximate value is acquired for the skin resistance value, and the reference resistor 38 having the most suitable resistance value is selected from among plural reference resistors 38 each time the bioelectrical impedance of skin is measured. In other words, the reference resistor 38 closest to the reference value obtained via this preliminary measurement is selected. The reference resistor 38 is used in a bioelectrical impedance measurement algorithm (described below) to calibrate the measurement system. Note that the sine wave generator circuit 30 used when step S4 is being executed is not limited to the sine wave generator circuit 30-1, and another suitable circuit may be selected from among the plural (four in the present exemplary embodiment) sine wave generator circuits in consideration of the measurement system.

In step S5, a sine wave is generated from the sine wave generator circuit 30-1.

In step S6, using the correlation value calculation circuit 31-1, the calculation of a correlation value between the sine wave signal output from the sine wave generator circuit 30-1 and the output (i.e. the measurement signal) from the ADC 33 is started. In other words, the frequency component of the sine wave generator circuit 30-1 is detected from the measurement signal.

In step S7, the process waits for the processing to end. In the present exemplary embodiment, ‘waiting for the processing to end’ refers to the next processing not being performed (i.e., the process waits on standby) until a predetermined measurement time (hereinafter, referred to as ‘wait time’) has elapsed. The predetermined wait time is set in accordance with the number of calculation samples and the like. In the present exemplary embodiment, the wait time is input via a UI section (not illustrated in the drawings) or the like. However, the present disclosure is not limited to this, and the wait time may instead be set within the present measurement program.

In step S8, the approximate resistance value of the skin is calculated using the correlation value.

Here, although it is an approximate value, the resistance value of the skin is actually calculated via the preliminary measurement. Therefore, for example, if only an approximate idea of the state of the skin is sought, and accuracy is not especially required, then the resistance value obtained via this preliminary measurement may be used as the resistance value of the skin.

As illustrated in FIG. 3, in step S9, the wait time is set. The processing after step S9 is the portion of the present measurement processing that is performed in order to actually measure the bioelectrical impedance of the skin.

In step S10, whether or not to use the sine wave generator circuit 30-1 is determined. The processing of this step is performed based on, for example, settings from a UI section (not illustrated in the drawings) or the like. If the result of this determination is affirmative, the process proceeds to step S11, while if the result is negative, the process proceeds to the subsequent step S12.

In step S11, the parameters of the sine wave generator circuit 30-1 are set. These parameters in the present exemplary embodiment are the oscillation frequency of the sine wave generator circuit 30-1, the reference frequency thereof, and the calculation sample number. Naturally, the parameters are not limited to these, and other parameters such as the amplitude and the like may also be set. The reference frequency is the reference frequency used when correlation values are calculated by the correlation value calculation circuits 31, and in the present exemplary embodiment, is the frequency of the corresponding sine wave generator circuit 30. The calculation sample number is the number of sample points when a correlation value is being calculated, and the above-described wait time is determined based on this sample number. The sample number may be the same for each of the sine wave generator circuits 30, or may be different for each sine wave generator circuit 30.

In step S12, in the same way as in step S10, whether or not to use the sine wave generator circuit 30-2 is determined, while in step S13, the parameters of the sine wave generator circuit 30-2 are set. In the same way, in steps S14 and S15, processing relating to the sine wave generator circuit 30-3 is executed, and in steps S16 and S17, processing relating to the sine wave generator circuit 30-4 is executed.

In step S18, the oscillation of the sine wave generator circuit 30 selected in step S10 through step S17 is started.

As illustrated in FIG. 4, in step S19, ending settings for the sine wave generator circuit 30 are made, and the calculation of the correlation value is started.

In step S20, the process waits on standby until the wait time has ended.

In step S21, whether or not the sine wave generator circuit 30-1 is to be used, in other words, whether or not the sine wave generator circuit 30-1 was selected in step S10 through step S17 is determined. If the result of this determination is affirmative, then in step S22, the correlation value calculation results are read from the correlation value calculation circuit 31-1. If, however, the result of the determination in step S21 is negative, the process proceeds to step S23.

In steps S23 and S24, the same type of processing as in steps S21 and S22 is performed for the sine wave generator circuit 30-2, while in steps S25 and S26, the same type of processing as in steps S21 and S22 is performed for the sine wave generator circuit 30-3, and in steps S27 and S28, the same type of processing as in steps S21 and S22 is again performed for the sine wave generator circuit 30-4. Note that, in the present exemplary embodiment, a case in which the determinations as to whether or not each of the sine wave generator circuits 30 has been selected are performed successively, has been described as an example. However, the present disclosure is not limited thereto. The selection results of steps S10 through S17 may be stored in advance in a storage device such as the RAM or the like (not illustrated in the drawings), and for processing that corresponds to steps S21 through S28 may be executed using these selection results.

In step S29, the measurement terminals are connected to the reference resistor. In other words, the switching circuits 36 are switched to the reference resistor 38.

In step S30, oscillation of the sine wave generator circuit 30 being used is enabled. In other words, oscillation from that sine wave generator circuit 30 is started.

In step S31, ending settings for the sine wave generator circuit 30 are made, and the calculation of the correlation value by the correlation value calculation circuit 31 is started.

In step S32, the process waits on standby until the wait time has ended. Thereafter, the switching circuits 36 are switched so that the skin is placed in contact with the device main body 10.

In step S33, whether or not the sine wave generator circuit 30-1 is to be used, in other words, whether or not the sine wave generator circuit 30-1 was selected in step S10 through step S17 is determined. If the result of this determination is affirmative, then in step S34, the correlation value calculation results are read from the correlation value calculation circuit 31-1. If, however, the result of the determination in step S33 is negative, the process proceeds to step S35.

In steps S35 and S36, the same type of processing as in steps S33 and S34 is performed for the sine wave generator circuit 30-2, while in steps S37 and S38, the same type of processing as in steps S33 and S34 is performed for the sine wave generator circuit 30-3, and in steps S39 and S40, the same type of processing as in steps S33 and S34 is again performed for the sine wave generator circuit 30-4. Note that, in the present exemplary embodiment, a case in which the determinations as to whether or not each of the sine wave generator circuits 30 has been selected are performed successively, has been described as an example. However, the present disclosure is not limited thereto. The selection results of steps S10 through S17 may be stored in advance in a storage device such as the RAM or the like (not illustrated in the drawings), and for processing that corresponds to steps S33 through S40 may be executed using these selection results.

In step S41, impedance values (i.e., resistance values and capacity values) are calculated for all frequencies, in other words, for each of the frequencies of the selected sine wave generator circuits 30. The calculations of these impedance values are performed by performing calculations using skin correlation values acquired via the above-described processing, and correlation values of the reference resistances.

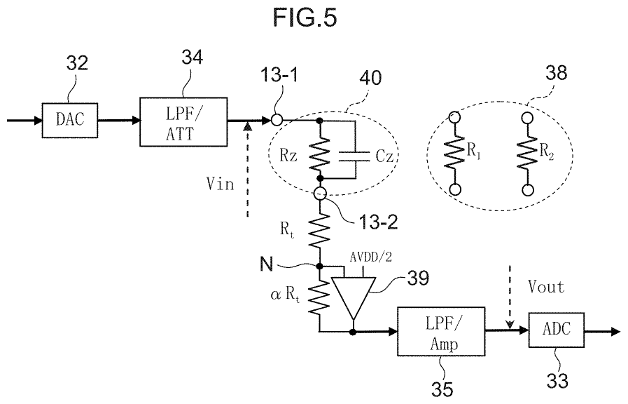

Next, a measurement algorithm for bioelectrical impedance executed by the measurement system 1 according to the present exemplary embodiment will be described with reference to FIG. 5.

FIG. 5 illustrates a calculation model (i.e., an equivalent circuit) referred to in the calculation algorithm described below. Each one of the DAC 32, LPF/ATT 34, LPF/Amp 35, ADC 33, and electrodes 13-1 and 13-2 illustrated in FIG. 5 is the same as those illustrated in FIG. 2.

A surface portion (i.e., a fold of skin) of a test subject is disposed between the electrodes 13-1 and 13-2 illustrated in FIG. 5 by being pinched between these, and, in the present example, a model of the skin (i.e., a skin model 40) is formed by parallel circuits (hereinafter, this parallel impedance is represented as ‘Z’ (i.e., a skin impedance), namely, a resistor Rz (i.e., a skin resistor) and a capacitor Cz (i.e., a skin capacitor). In the present exemplary embodiment, two resistors having respective resistance values of R1 and R2 (referred to on occasion respectively as a ‘reference resistor R1’ and a ‘reference resistor R2’) are used as the reference resistor 38. Naturally, the reference resistor 38 is not limited to two resistors, and three or more reference resistors 38 may be used depending on the state of the skin and the like. Rt illustrated in FIG. 5 is an input resistor (in other words, an input resistor of a measurement instrument) of an operational amplifier 39 (RAMP), α is the gain-phase ratio (i.e., of the measurement device) relative to the input resistor Rt. The operational amplifier 39 sets an operating point (denoted as ‘AVDD/2’ in FIG. 5) of a measurement signal input from the electrode 13-2, and an operating point of a node N is set by these.

In FIG. 5, a measurement signal output from the LPF/ATT 34 is denoted as ‘Vin’, while a measurement signal transmitted through a test subject and output from the LPF/Amp 35 is denoted as ‘Vout’. In other words, Vin is an addition of the effect of the LPF/ATT 34 and the output from the DAC 32, while Vout is an addition of the effect of the LPF/Amp 35 and the output from the electrode 13-2. The phase and gain effects of the Vin system and the Vout system are aggregated in a. The characteristics of the LPF/Amp 35 on the receiving side as well are also grouped together in a. Moreover, in the present exemplary embodiment, the Vin system and the Vout system are separated by the operational amplifier (RAMP) 39.

In accordance with the above modeling, in a case in which the impedance Z of the skin model is determined as Z=Re+Im, then a resistance value R_{z} and a capacity value C_{z} of the skin model are calculated in accordance with Formula (1) through Formula (3) shown below. Note that R is a real part of Z, while Im is an imaginary part of Z.

$$\begin{matrix}{R}_{Z} = \frac{{R}_{e}^{2} + {I}_{m}^{2}}{{R}_{e}} & ( 1 ) \\ {C}_{Z} = \frac{{I}_{m}}{ω ( {R}_{e}^{2} + {I}_{m}^{2} )} & ( 2 ) \\ {I}_{m} = ω {C}_{Z} ( {R}_{e}^{2} + {I}_{m}^{2} ) & ( 3 )\end{matrix}$$

Hereinafter, the derivation of Formula (1) through Formula (3) will be described in detail. The output Vout used in the following calculation is defined as follows.

V_{oR1}: Output Vout when the reference resistor R1 is connected

V_{oR2}: Output Vout when the reference resistor R2 is connected

V_{oZ}: Output Vout when the skin is connected

At this time, V_{oR1}, V_{oR2}, and V_{oZ} are expressed as is shown below in Formula (4).

$$\begin{matrix}{V}_{OR 1} = \frac{α {R}_{t}}{R 1 + {R}_{t}} \cdot {V}_{in} \\ {V}_{OR 2} = \frac{α {R}_{t}}{R 2 + {R}_{t}} \cdot {V}_{in} \\ {V}_{OZ} = \frac{α {R}_{t}}{Z + {R}_{t}} \cdot {V}_{in}\end{matrix} \} \qquad ( 4 )$$

(1) Calculation of Rₜ

The following formula is obtained by transforming Formula (4).

$$\begin{matrix}{V}_{oR 1} ( R 1 + {R}_{t} ) = α {R}_{t} \cdot {V}_{in} \\ {V}_{oR 2} ( R 2 + {R}_{t} ) = α {R}_{t} \cdot {V}_{in}\end{matrix} \}$$

If the formula is transformed so that the two left sides are mutually equivalent, then Rₜ can be calculated in accordance with Formula (5) shown below.

$${V}_{oR 1} ( R 1 + {R}_{t} ) = {V}_{oR 2} ( R 2 + {R}_{t} ) \\ {V}_{oR 1} R 1 + {V}_{oR 1} {R}_{t} = {V}_{oR 2} R 2 + {V}_{oR 2} {R}_{t} \\ ( {V}_{oR 1} - {V}_{oR 2} ) {R}_{t} = {V}_{oR 2} R 2 - {V}_{toR 1} R 1 \\ {R}_{t} = \frac{{V}_{oR 2} R 2 - {V}_{oR 1} R 1}{{V}_{oR 1} - {V}_{oR 2}} \qquad ( 5 )$$

(2) Calculation of Vin

Since Vin is not a direct output from the DAC 32, it must be arithmetically calculated.

(2-1) Calculation from V_{oR1}

The following formula is obtained by transforming Formula (4).

$${V}_{in} = \frac{R 1 + {R}_{t}}{α {R}_{t}} \cdot {V}_{oR 1}$$

If the above formula is transformed by the substitution of Formula (5), then Vin is calculated using V_{oR1} in accordance with Formula (6) shown below.

$${V}_{in} = \frac{R 1 + \frac{{V}_{oR 2} R 2 - {V}_{oR 1} R 1}{{V}_{oR 1} - {V}_{oR 2}}}{α \cdot \frac{{V}_{oR 2} R 2 - {V}_{oR 1} R 1}{{V}_{oR 1} - {V}_{oR 2}}} \cdot {V}_{oR 1} \\ {V}_{in} = \frac{R 1 ( {V}_{oR 1} - {V}_{oR 2} ) + ( {V}_{oR 2} R 2 - {V}_{oR 1} R 1 )}{α \cdot ( {V}_{oR 2} R 2 - {V}_{oR 1} R 1 )} \cdot {V}_{oR 1} \\ {V}_{in} = \frac{R 1 \cdot {V}_{oR 1} - R 1 \cdot {V}_{oR 2} + ( {V}_{oR 2} R 2 - {V}_{oR 1} R 1 )}{α \cdot ( {V}_{oR 2} R 2 - {V}_{oR 1} R 1 )} \cdot {V}_{oR 1} \\ {V}_{in} = \frac{R 2 \cdot {V}_{oR 2} - R 1 \cdot {V}_{oR 2}}{α \cdot ( R 2 \cdot {V}_{oR 2} - R 1 \cdot {V}_{oR 1} )} \cdot {V}_{oR 1} \qquad ( 6 )$$

(2-2) Calculation from V_{oR2}

The following formula is obtained by transforming Formula (4).

$${V}_{in} = \frac{R 2 + {R}_{t}}{α {R}_{t}} \cdot {V}_{oR 2}$$

If the above formula is transformed by the substitution of Formula (5), then Vin is calculated using V_{oR2} in accordance with Formula (7) shown below.

$${V}_{in} = \frac{R 2 + \frac{{V}_{oR 2} R 2 - {V}_{oR 1} R 1}{{V}_{oR 1} - {V}_{oR 2}}}{α \cdot \frac{{V}_{oR 2} R 2 - {V}_{oR 1} R 1}{{V}_{oR 1} - {V}_{oR 2}}} \cdot {V}_{oR 2} \\ {V}_{in} = \frac{R 1 ( {V}_{oR 1} - {V}_{oR 2} ) + ( {V}_{oR 2} R 2 - {V}_{oR 1} R 1 )}{α \cdot ( {V}_{oR 2} R 2 - {V}_{oR 1} R 1 )} \cdot {V}_{oR 2} \\ {V}_{in} = \frac{R 2 \cdot {V}_{oR 1} - R 2 \cdot {V}_{oR 2} + ( {V}_{oR 2} R 2 - {V}_{oR 1} R 1 )}{α \cdot ( {V}_{oR 2} R 2 - {V}_{oR 1} R 1 )} \cdot {V}_{oR 2} \\ {V}_{in} = \frac{R 2 \cdot {V}_{oR 1} - R 1 \cdot {V}_{oR 1}}{α \cdot ( R 2 \cdot {V}_{oR 2} - R 1 \cdot {V}_{oR 1} )} \cdot {V}_{oR 2} \qquad ( 7 )$$

(3) Calculation of Z

The following formula is obtained by transforming Formula (4).

$${V}_{oZ} = \frac{α {R}_{t}}{Z + {R}_{t}} \cdot {V}_{in}$$

$${V}_{oZ} \cdot ( Z + {R}_{t} ) = α {R}_{t} \cdot {V}_{in}$$

$$Z = \frac{α {R}_{t} \cdot {V}_{in}}{{V}_{oZ}} - {R}_{t}$$

(3-1) Calculation from V_{oR1}

If the above formula is transformed by the substitution of the Rₜ from Formula (5) and the Vin from Formula (6), then Z is calculated in accordance with Formula (8) shown below.

$$Z = \frac{α ( \frac{{V}_{oR 2} R 2 - {V}_{oR 1} R 1}{{V}_{oR 1} - {V}_{oR 2}} ) \cdot ( \frac{R 2 \cdot {V}_{oR 2} - R 1 \cdot {V}_{oR 2}}{α \cdot ( R 2 \cdot {V}_{oR 2} - R 1 \cdot {V}_{oR 1} )} \cdot {V}_{oR 1} )}{{V}_{oZ}} - \frac{{V}_{oR 2} R 2 - {V}_{oR 1} R 1}{{V}_{oR 1} - {V}_{oR 2}} \\ Z = \frac{( R 2 \cdot {V}_{oR 2} - R 1 \cdot {V}_{oR 2} ) \cdot {V}_{oR 1} - ( R 2 \cdot {V}_{oR 2} - R 1 \cdot {V}_{oR 1} ) \cdot {V}_{oZ}}{( {V}_{oR 1} - {V}_{oR 2} ) \cdot {V}_{oZ}} \qquad ( 8 )$$

(3-2) Calculation from V_{oR2}

If the above formula is transformed by the substitution of the Rₜ from Formula (5) and the Vin from Formula (7), then Z is calculated in accordance with Formula (9) shown below.

$$Z = \frac{α ( \frac{{V}_{oR 2} R 2 - {V}_{oR 1} R 1}{{V}_{oR 1} - {V}_{oR 2}} ) \cdot ( \frac{R 2 \cdot {V}_{oR 1} - R 1 \cdot {V}_{oR 1}}{α \cdot ( R 2 \cdot {V}_{oR 2} - R 1 \cdot {V}_{oR 1} )} \cdot {V}_{oR 2} )}{{V}_{oZ}} - \frac{{V}_{oR 2} R 2 - {V}_{oR 1} R 1}{{V}_{oR 1} - {V}_{oR 2}} \\ Z = \frac{( R 2 \cdot {V}_{oR 1} - R 1 \cdot {V}_{oR 1} ) \cdot {V}_{oR 2} - ( R 2 \cdot {V}_{oR 2} - R 1 \cdot {V}_{oR 1} ) \cdot {V}_{oZ}}{( {V}_{oR 1} - {V}_{oR 2} ) \cdot {V}_{oZ}} \qquad ( 9 )$$

Here, the Re and Im of the above-described skin model satisfy the following formula.

$$Z = \frac{1}{\frac{1}{{R}_{Z}} + j ω {C}_{Z}} = \frac{{R}_{Z}}{1 + j ω {C}_{Z} {R}_{Z}} = \frac{{R}_{Z} - j ω {C}_{Z} {R}_{Z}^{2}}{1 + {ω}^{2} {C}_{Z}^{2} {R}_{Z}^{2}}$$

$${R}_{e} = \frac{{R}_{Z}}{1 + {ω}^{2} {C}_{Z}^{2} {R}_{Z}^{2}} , {I}_{m} = - \frac{ω {C}_{Z} {R}_{Z}^{2}}{1 + {ω}^{2} {C}_{Z}^{2} {R}_{Z}^{2}}$$

$$\frac{{I}_{m}}{{R}_{e}} = \frac{\frac{- ω {C}_{Z} {R}_{Z}^{2}}{1 + {ω}^{2} {C}_{Z}^{2} {R}_{Z}^{2}}}{\frac{{R}_{Z}}{1 + {ω}^{2} {C}_{Z}^{2} {R}_{Z}^{2}}} = - ω {C}_{Z} {R}_{Z}$$

$$\frac{{I}_{m}^{2}}{{R}_{e}^{2}} = {ω}^{2} {C}_{Z}^{2} {R}_{Z}^{2}$$

If the relationship shown by the above formula is substituted into the formula of Re, then the resistance value R_{Z} and the capacity value C_{Z} are calculated using Formula (1) and Formula (2) which are reproduced below.

$$\begin{matrix} {R}_{e} = \frac{{R}_{Z}}{1 + \frac{{I}_{m}^{2}}{{R}_{e}^{2}}} = \frac{{R}_{Z} {R}_{e}^{2}}{{R}_{e}^{2} + {I}_{m}^{2}} \\ {R}_{Z} = \frac{{R}_{e}^{2} + {I}_{m}^{2}}{{R}_{e}} & ( 1 ) \\ {I}_{m} = - \frac{ω {C}_{Z} {R}_{Z}^{2}}{1 + \frac{{I}_{m}^{2}}{{R}_{e}^{2}}} = \frac{ω {C}_{Z} {R}_{Z}^{2} {R}_{e}^{2}}{{R}_{e}^{2} + {I}_{m}^{2}} = \frac{ω {C}_{Z} {{R}_{e}^{2} ( \frac{{R}_{e}^{2} + {I}_{m}^{2}}{{R}_{e}} )}^{2}}{{R}_{e}^{2} + {I}_{m}^{2}} = ω {C}_{Z} ( {R}_{e}^{2} + {I}_{m}^{2} ) & ( 2 ) \\ \frac{1}{ω {C}_{Z}} = \frac{{R}_{e}^{2} + {I}_{m}^{2}}{{I}_{m}} \\ {C}_{Z} = \frac{{I}_{m}}{ω ( {R}_{e}^{2} + {I}_{m}^{2} )} & ( 3 )\end{matrix}$$

As shown above, the gain-phase ratio α is cancelled out in the calculation process, and is not included in Formula (1) through Formula (3).

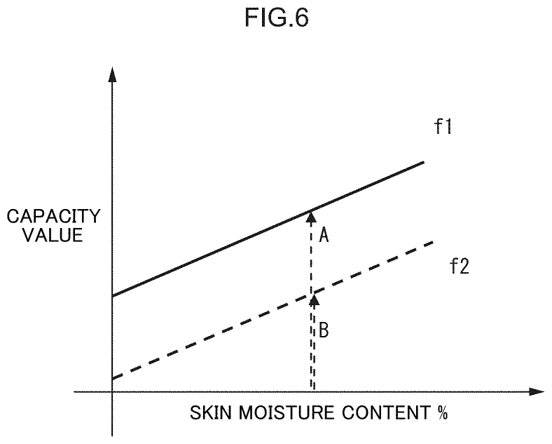

Next, a calculation of capacity values using the semiconductor device, measurement system, and measurement method according to the present exemplary embodiment will be described in more detail with reference to FIG. 6. By using the above-described measurement system, calculation algorithm, and calculation model, in the semiconductor device, measurement system, and measurement method according to the present exemplary embodiment, for example, an impedance, resistance value, and capacity value are acquired simultaneously and via the same electrodes 13 for each different frequency of skin.

Here, at approximately 80, the relative dielectric constant of water is extremely high. In contrast, the capacity value of skin varies greatly depending on the moisture content of that skin. Furthermore, the relative dielectric constant of water is also affected by the frequency of the measurement signal (i.e., the measurement current)

FIG. 6 illustrates a relationship between a capacity value of skin and a moisture rate of skin (as a percentage; denoted as ‘skin moisture content %’ in FIG. 6). In FIG. 6, the solid line illustrates a change in the skin capacity value relative to the skin moisture content percentage when the frequency is f1 (for example, 5 kHz), while the broken line illustrates a change in the skin capacity value relative to the skin moisture content percentage when the frequency is f2 (greater than f1; for example, 30 kHz).

If the capacity values for a skin moisture content percentage of a particular value when the frequencies are f1 and f2 are taken respectively as A and B, then the value of B/A has a correlation with the skin moisture content percentage. In other words, the skin moisture content percentage can be obtained from the value of B/A. In addition, generally, the capacity value varies greatly depending on the size of the electrodes. However, in the semiconductor device, measurement system, and measurement method according to the present exemplary embodiment, because the measurements are made simultaneously using the same electrodes 13, the difference between the surface areas pinched by the electrodes is expressed, for example, by a multiplier k. In other words, if the capacity values at the frequencies f1 and f2 when, for example, the electrodes 13 are reattached are taken as A′ and B′, then A′=k·A, and B′=k·B. In this case,

B′/A′=(k·B)/(k·A)=B/A

is established, and the ratio of the capacities is not affected by variations between the surface areas pinched by the electrodes 13. Because there is no effect from the variations between the surface areas pinched by the electrodes 13, even if the electrodes 13 are formed in a spherical shape, as is illustrates in FIG. 1, so that they are able to pinch a fold of skin between them, it is still possible to obtain an accurate bioelectrical impedance value (i.e., a resistance value and a capacity value). As a result, the semiconductor device, measurement system, and measurement method according to the present exemplary embodiment make it possible to measure deep portions of skin that it has not, hitherto, been possible to measure using electrodes according to the conventional technology. Note that the above description is also valid with regard to the measurement of resistance values, and the measurement of resistance values is also not affected by variations in the surface area pinched between electrodes. In addition, an example in which measurements are made using a skin sensor has been described above, however, it is also possible to measure body fat as well using the above-described principle without these body fat measurements being affected by the surface area pinched between the electrodes.

As has been described above, according to the semiconductor device, measurement system, and measurement method of the present exemplary embodiment, the shape of the electrodes can be freely designed. Accordingly, a body composition measurement device that may pinch a skin may be provided, which has not hitherto been possible since the surface area of the electrodes may change. According to the present exemplary embodiment, the skin sensor of the present disclosure may be incorporated in a facial roller, to obtain information (such as the collagen content and the like) about deep portions of skin.

Moreover, although the semiconductor device, measurement system, and measurement method according to the present exemplary embodiment are suitable for skin sensors and body fat measurement, since the semiconductor device, measurement system, and measurement method of the present disclosure may detect variations in the frequency characteristics of a body composition, if the relationship between frequency and body composition, for example, the frequency of collagen, or the frequency of each type of cellulite are used, then they may also be used as a comprehensive skin sensor device that measures specific details of the state of skin health.

Furthermore, according to the semiconductor device, measurement system, and measurement method according to the present exemplary embodiment, by using a skin care device of a type that pinches a skin between spherical electrodes, the body composition measurement may be performed continuously even during skin care treatment. Since the present exemplary embodiment may continuously measure how the skin changes during skin care treatment, the present exemplary embodiment may, for example, determine at what point the skin care treatment should end. In other words, according to the semiconductor device, measurement system, and measurement method according to the present exemplary embodiment, a skin care device and a biological composition measurement device may be integrated into a single device. Namely, during skin care treatment in which a micro current is supplied to a test subject, if the micro current is supplied from the device main body 10 to spherical electrodes that are capable of rotating, then it is possible to form a structure in which this micro current and measurement current for the biological composition measurement are supplied on a time division basis. Exemplary embodiments of the present disclosure has been described above, however, the present disclosure is not limited to this. Various modifications and the like may be made to the present disclosure insofar as they do not depart from the spirit or scope of the present disclosure.

What is claimed is:

1. A semiconductor device comprising:
a signal source that generates a sine wave signal;
an output section that outputs a measurement signal corresponding to the sine wave signal to a test subject via a first electrode;
an input section that receives, as an input signal, the measurement signal that has passed through the test subject and been input via a second electrode;
a first calculation device that calculates correlation values between the sine wave signal and the input signal; and
a second calculation section that, based on the correlation values, calculates a bioelectrical impedance of the test subject.

2. The semiconductor device according to claim 1, wherein:
the signal source includes a plurality of the signal sources that respectively generate sine wave signals having mutually different frequencies,
the semiconductor device further comprises an adding section that adds the sine wave signals generated by the plurality of signal sources,
the output section outputs, to the test subject, measurement signals corresponding to the sine wave signals added by the adding section,
the first calculation device calculates correlations between each one of the plurality of sine wave signals and the input signal, and
the second calculation device calculates the bioelectrical impedance based on the plurality of correlation values.

3. The semiconductor device according to claim 2, wherein the second calculation device further separates the biological impedance into a resistance component and a capacity component.

4. The semiconductor device according to claim 3, wherein the second calculation device measures a state of the body composition of the test subject, using a ratio of the resistance components or a ratio of the capacity components measured using the sine wave signals of two of the frequencies.

5. The semiconductor device according to claim 3, wherein the second calculation device measures a skin moisture content of the test subject using the ratio of the capacity components measured using the sine wave signals of two of the frequencies.

6. The semiconductor device according to claim 1, wherein:
the first calculation device additionally calculates correlation values between the sine wave signals and a reference resistance, and
in a case in which the second calculation device is calculating the bioelectrical impedance of the test subject based on the correlation values, the second calculation device calibrates a measurement system for the bioelectrical impedance using the correlation values of the reference resistance.

7. The semiconductor device according to claim 6, wherein;
the first calculation device calculates correlation values for two reference resistances; and
the second calculation device calibrates the measurement system for the bioelectrical impedance using the two reference resistance correlation values.

8. The semiconductor device according to claim 6, wherein:
the first calculation device calculates correlation values between the sine wave signals and the input signals using one of the signal sources, and
the second calculation device calculates a resistance value of the test subject based on these correlation values, and selects a value of the reference resistance using the resistance value.

9. A measurement system comprising:
the semiconductor device according to claim 1,
a first electrode that, outputs to a test subject, a measurement signal corresponding to the sine wave signal from the output section; and
a second electrode that receives, as an input signal, the measurement signal that has passed through the test subject.

10. The measurement system according to claim 9, wherein the first electrode and the second electrode are attached to the test subject by pinching a surface portion of the test subject.

11. The measurement system according to claim 10, wherein each of the first electrode and the second electrode has a spherical shape.

12. A measurement method that uses a measurement system including a plurality of signal sources that respectively generate sine wave signals having mutually different frequencies, a first electrode that outputs, to a test subject, a measurement signal corresponding to a signal obtained by adding a plurality of the sine wave signals, and a second electrode that receives, as an input signal, the measurement signal that has passed through the test subject, the measurement method comprising:
calculating correlation values between each one of the sine wave signals and the input signal;
calculating a bioelectrical impedance of the test subject using the plurality of correlation values;
separating the bioelectrical impedance into a resistance component and a capacity component; and
measuring at least one of a state of the body composition of the test subject, by using a ratio of the resistance components, or a ratio of the capacity components measured by using each one of two frequencies, or a skin moisture content of the test subject, by using the ratio of the capacity components measured by using each one of the two frequencies.
